(19) United States
(12) Patent Application Publication
(10) Pub. No.: US 2014/0316370 A1
(43) Pub. Date: Oct. 23, 2014

(54) COMBINATION OF A DRIP CHAMBER FOR USE IN A DRIP INFUSION SET

(71) Applicant: DRIPMATE A/S, Charlottenlund (DK)

(72) Inventors: Morten Mernoe, Charlottenlund (DK); Morten Think, Espegaerde (DK)

(73) Assignee: DRIPMATE A/S, Charlottenlund (DK)

(21) Appl. No.: 14/358,916

(22) PCT Filed: Oct. 31, 2012
(86) PCT No.: PCT/DK2012/050403

§ 371 Date: May 16, 2014

(30) Foreign Application Priority Data
Nov. 18, 2011 (DK) PA 2011 00907

(51) Int. Cl. A61M 5/14; A61M 5/168

(52) U.S. Cl. CPC A61M 5/1411; A61M 5/1689; A61M 2205/3306; A61M 2205/3317; A61M 2205/6027; A61M 2205/6036; A61M 2205/6045; A61M 2205/6054; A61M 2205/6063; A61M 2205/6072; A61M 2205/6081; A61M 2205/3584
USPC 604/500; 604/253

(57) ABSTRACT

There is provided a combination of a drip chamber for use in a drip infusion set of the type comprising a liquid supply connected to said drip chamber upstream thereof and an injection needle connected through a tube to said drip chamber downstream thereof, and a drop sensor adapted for being arranged adjacent said drip chamber for sensing the passage of drops through said drip chamber.

The present invention relates to a combination of a drip chamber for use in a drip infusion set of the type comprising a liquid supply connected to said drip chamber upstream thereof and an injection needle connected through a tube to said drip chamber downstream thereof, and a drop sensor adapted for being arranged adjacent said drip chamber for sensing the passage of drops through said drip chamber.

In connection with such combinations there is a danger that a specific drop sensor is not compatible with a specific drip chamber which can entail health endangering errors in the treatment of patients being treated with such combinations.

Thus, a main object of the invention is to provide means for preventing errors deriving from incompatibility between said drip chamber and said drop sensor.

Furthermore, it is an object of the invention to enhance the functionality of a drip infusion set incorporating a drip chamber and a drop sensor.

According to the invention, this object is attained by said drip chamber being provided with characterization means for characterizing said drip chamber, and said drop sensor being provided with corresponding identification means for identifying and recognizing said characterization means.

According to the invention, the characterization means and the corresponding identification means may be chosen from a group of characterization means and corresponding identification means comprising:
a) a specific drip chamber surface, and
   a corresponding drop chamber surface adapted for engaging said drip chamber surface,
said drip chamber surface deviating from a circular cylindrical configuration,
b) a specifically shaped protuberance of one of said drip chamber and said drop sensor and
   a correspondingly shaped recess of the second of said drip chamber and said drop sensor adapted for receiving said protuberance,
c) an electrically conductive element forming part of said drip chamber and having a specific resistance between first two electrical contacts thereof and
   corresponding second electrical contacts forming part of said drop sensor,
said drop sensor being provided with means to measure said resistance when said first and second contacts are interconnected,
d) an electrical circuit containing specific information regarding said drip chamber and forming part of said drip chamber, and
   sensing means for sensing said specific information and forming part of said drop sensor,
e) an RFID tag containing specific information regarding said
   RFID sensing means for sensing said RFID tag and forming part of said drop sensor,
f) a bar code containing specific information regarding said drip chamber and forming part of said drip chamber, and
   a bar code reader for reading said bar code and forming part of said drop sensor,
g) a QR code containing specific information regarding said drip chamber and forming part of said drip chamber, and
   image generating means such as a camera for generating a digital image of said QR code and forming part of said drop sensor,
h) two capacitor electrodes constituting a capacitor element and forming part of said drop sensor, and
   a corresponding electrically conductive element forming part of said drip chamber and adapted for being located between said capacitor electrodes when said drop sensor engages said drip chamber such that the electrical capacity of said capacitor element depends on the configuration of said electrically conductive element when said electrically conductive element is located between said two capacitor electrodes.
said drop sensor being provided with means for measuring said electrical capacity, and
i) visually identifiable means such as shape, colour, markings and the like forming part of said drip chamber, and
   image generating means such as a camera for generating a digital image of said visually identifiable means and forming part of said drop sensor.

Hereby, compatibility between the drip chamber and the corresponding drop sensor is ensured and, by allowing information to be interchanged between the drip chamber and the drop sensor, enhancement of the functionality of the drip infusion set incorporating these features is made possible.

In another aspect, the invention relates to a drip chamber for use in a drip infusion set of the type comprising a liquid supply connected to said drip chamber upstream thereof and an injection needle connected to said drip chamber downstream thereof, said drip chamber being provided with the characterization means specified above, wherein the outer surface of said drip chamber surface deviates from a circular cylindrical configuration.

In a further aspect, the invention relates to a drop sensor adapted for being arranged adjacent a drip chamber for use in a drip infusion set of the type comprising a liquid supply connected to said drip chamber upstream thereof and an injection needle connected to said drip chamber downstream thereof, said drop sensor being provided with the identification means specified above

Preferably, the combination according to the invention may further comprise a liquid flow control device comprising a valve adapted for controlling the flow of said liquid through said tube.

In a yet further aspect, the invention relates to a liquid flow control system for use with drip infusion sets of the type comprising a liquid supply, a drip chamber downstream of the liquid supply for forming liquid drops and a tube connecting said drip chamber with an injection needle, said system comprising:
a drop sensor adapted for being arranged adjacent said drip chamber for sensing the passage of drops through said drip chamber,
a valve adapted for controlling the flow of said liquid through said tube,
valve activating means for activating said valve,
electrical circuits for registering the rate of drop formation and controlling said valve activating means,
a battery for powering said drop sensor, said electrical circuits and said valve activating means, and
identification means for identifying features relevant to the correct functioning of said system.

According to the invention said identification means may be chosen from a group of identification means comprising:
A) image generating means such as a digital camera for generating a digital image,
B) a bar code reader for reading a bar code, and
C) RFID sensing means for sensing an RFID tag.
D) Means to measure a resistance, and
E) Means to measure an electrical capacity.

Furthermore, according to the invention said features may be chosen from a group of features comprising:
1) a colour code on the drip chamber,
2) markings on the drip chamber,
3) a specific shape of the drip chamber,
4) a resistance in an electrical conductor forming part of said drip chamber,
5) an electrical capacity in a capacitor forming part of said drip chamber,
6) an RFID tag,
7) a bar code,
8) a QR code, and
9) printed information.

Advantageously, the system may further comprise wire-less communication means.

Hereby, a one-way or two way interchange of information may take place based on information provided by said features and feed-back to the system from data-bases, the responsible doctor etc.

In yet another aspect, the invention relates to a method of ensuring correct function of a drip infusion set of the type comprising a liquid supply connected to a drip chamber upstream thereof and an injection needle connected to said drip chamber downstream thereof as well as a drop counter for sensing the passage of drops through said drip chamber for counting the drops in said drip chamber, said method comprising the following steps:
a) providing:
   a valve adapted for controlling the flow of said liquid through said tube,
   valve activating means for activating said valve,
   electrical circuits for registering the rate of drop formation and controlling said valve activating means,
   a battery for powering said drop sensor, said electrical circuits and said valve activating means,
b) providing said drip chamber with characterization means as specified above,
c) providing said drop sensor with identification means as specified above,
d) transmitting data generated by said identification means to said electrical circuits, and
e) causing said electrical circuits to control said valve activating means based on said data.

Advantageously, the method may comprise the further steps of:
a) providing wire-less communication means, and
b) causing said wire-less communication means to transmit and receive data including said data generated by said identification means.

In the following the invention will be described in more detail with reference to the accompanying drawings illustrating, solely by way of example, various embodiments of the invention, where

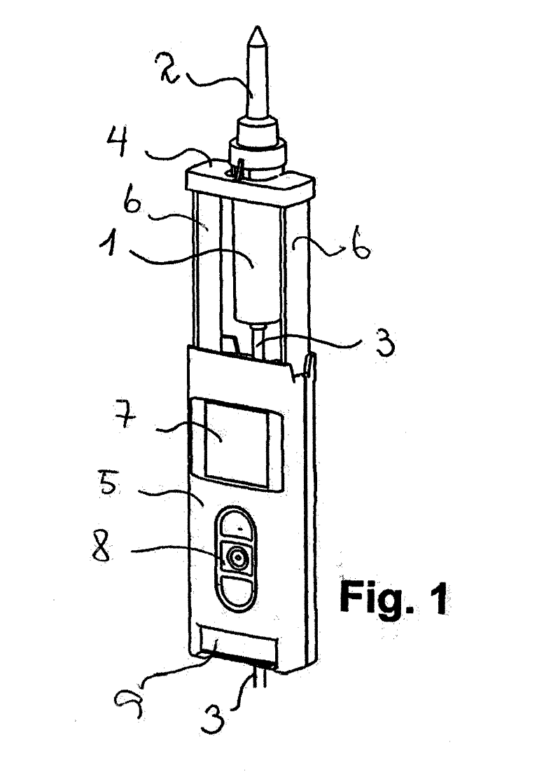

FIGS. 1-3 are schematic, perspective views of three embodiments of a drip infusion set liquid flow control system for use with a combination of drip chamber and drop sensor according to the invention,

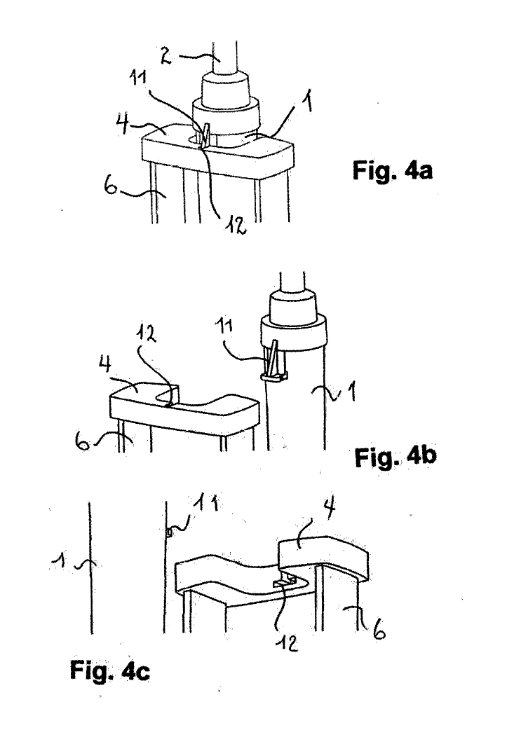

FIGS. 4a-4c are schematic cut-away views of a first embodiment of a combination of drip chamber and drop sensor according to the invention,

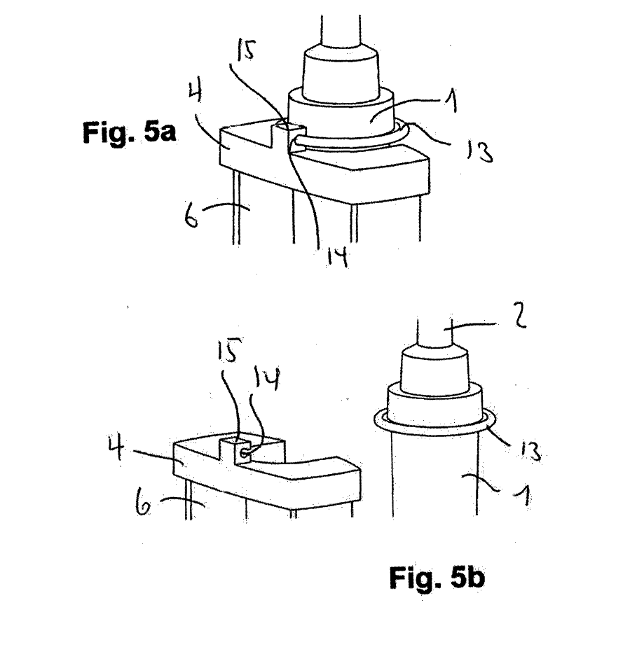

FIGS. 5a-5b are schematic cut-away views of a second embodiment of a combination of drip chamber and drop sensor according to the invention,

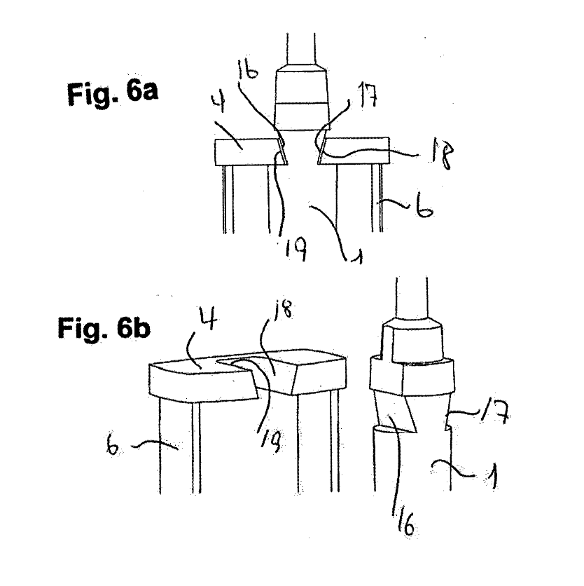

FIGS. 6a-6b are schematic cut-away views of a third embodiment of a combination of drip chamber and drop sensor according to the invention,

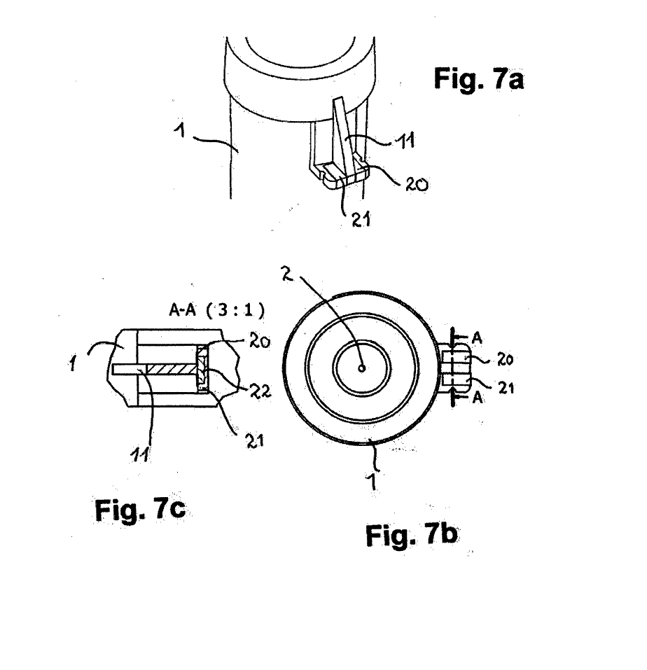

FIG. 7a is a schematic cut-away view of a fourth embodiment, related to the first embodiment of FIGS. 4a-4c,

FIG. 7b is a top view of the embodiment shown in FIG. 7a,

FIG. 7c is a cross sectional view in larger scale along line A-A in FIG. 7b,

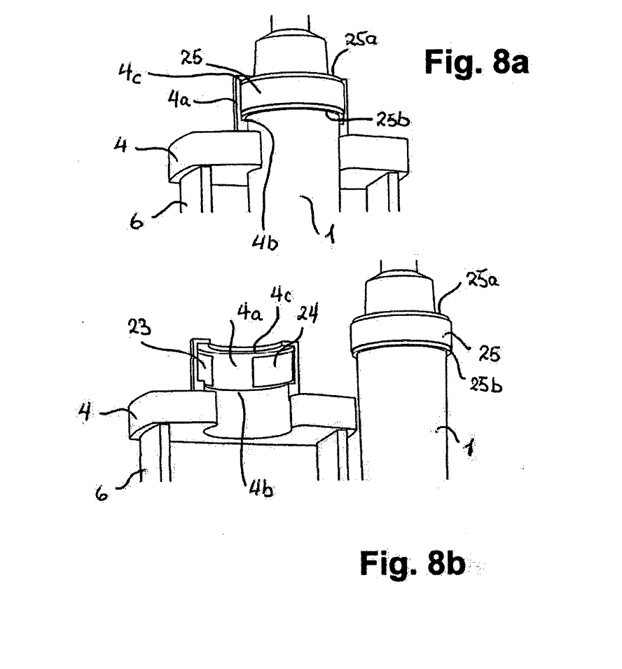

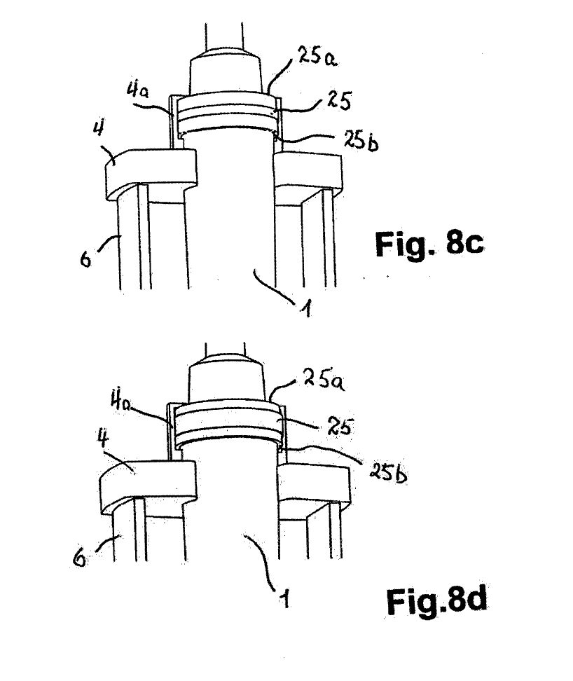

FIGS. 8a-8b are schematic cut-away views of a fifth embodiment of a combination of drip chamber and drop sensor according to the invention,

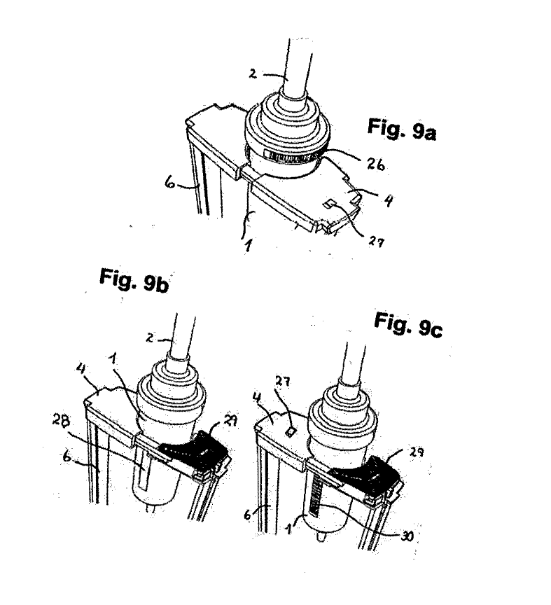

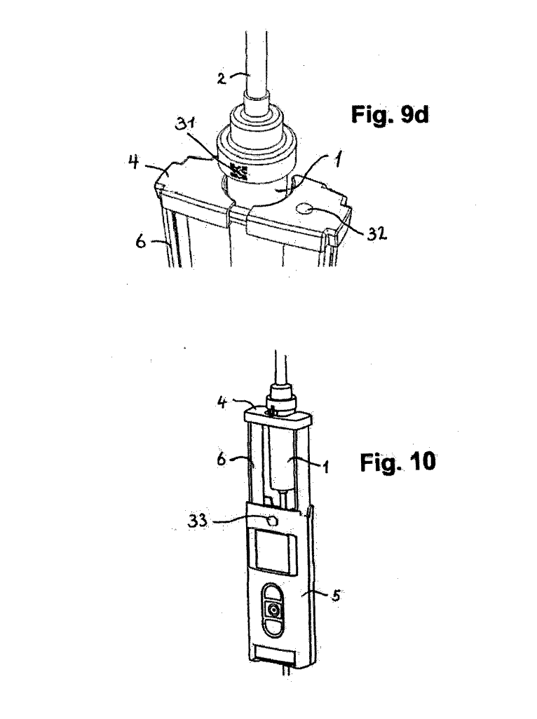

FIGS. 9a-9d are schematic cut-away views of sixth to ninth embodiments of a combination of drip chamber and drop sensor according to the invention, and

FIG. 10 shows a schematic perspective view of an embodiment of a drip infusion set liquid flow control system according to the invention.

Referring now to FIG. 1, a drip chamber 1 is connected to a not shown infusion liquid supply by a tube 2 and to a not shown injection needle through a tube 3.

A drop sensor 4 is arranged adjacent the drip chamber 1 such that drops falling through the drip chamber 1 may be sensed by the drop sensor 4.

A flow control device 5 is detachably attached to the tube 3 by means of not shown attachment means. The drop sensor is carried by the device 5 by means of telescopic arms 6 retractably arranged in the housing of device 5 that includes a display 7, control buttons 8 and a removable monitoring device 9 for remotely monitoring the operation of the flow control system shown in FIG. 1. The drop sensor is electrically connected to the device by means of a not shown electrical conductor.

The flow control system shown in FIG. 1 is described more in detail in U.S. Pat. No. 7,918,834 and US patent application No. 2011/0221596 A1, the disclosure of both of which is hereby incorporated herein by reference.

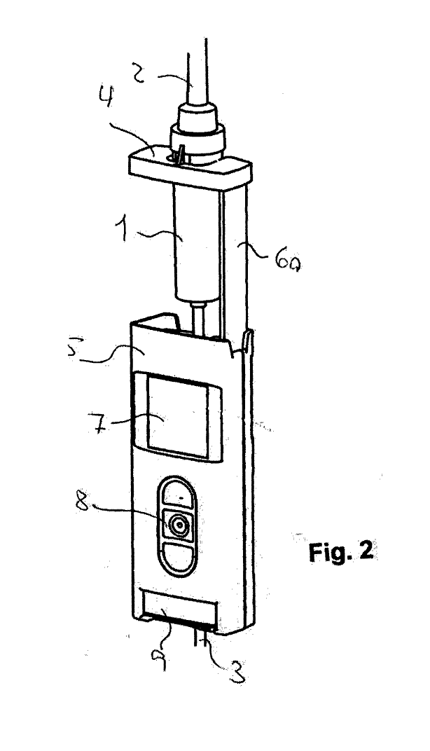

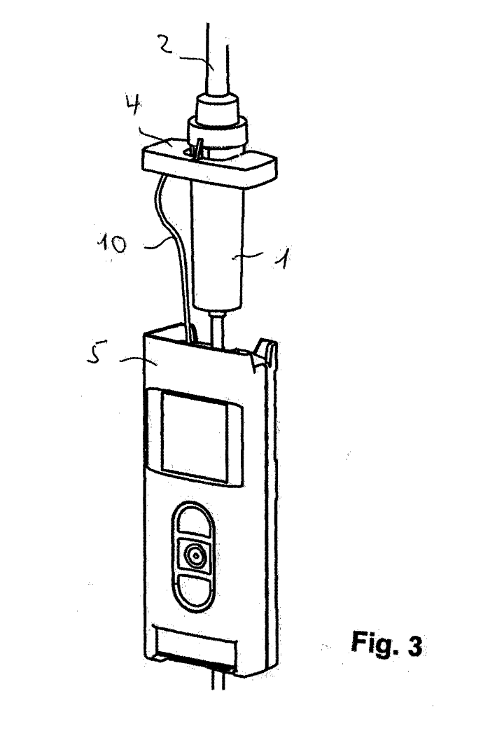

Referring now to FIGS. 2-3, the flow control systems shown therein are identical to the system shown in FIG. 1 except that in FIG. 2 only on telescopic arm 6a carries the drop sensor 4, and in FIG. 3 the sensor 4 is attached to the drip chamber 1 by means of not shown spring means and the sole connection between sensor 4 and device 5 is an electrical conductor 10.

Referring now to FIGS. 4a-4c, the manner of attachment of the drop sensor 4 to the drip chamber 1 is illustrated. A generally T-shaped protuberance 11 on the surface of the drip chamber 1 is inserted in a correspondingly shaped recess 12 in the surface of the drop sensor 4 whereby the drop sensor 4 is fixated in correct position relative to the drip chamber 1.

Referring now to FIGS. 5a-5b, the fixation of drop sensor 4 to drip chamber takes place by inserting a ring-shaped protuberance 13 in a cylindrical recess 14 in a portion 15 of the drop sensor whereby the drop sensor can be attached to the drip chamber in an axially predetermined position but with no circumferential fixation which renders it easier to attach the sensor 4 to the drip chamber.

Referring now to FIGS. 6a-6b, the fixation of the drop sensor 4 on the drip chamber takes place by inserting a narrow neck portion of the drip chamber formed by opposed recesses 16 and 17 in a correspondingly shaped recess in the drop sensor 4 formed by opposed surfaces 18 and 19.

Referring now to FIGS. 7a-7c, the protuberance 11 shown in FIGS. 4a-4c is provided with two embedded electrodes 20, 21 with a resistance element 22 with a specific resistance connecting them electrically. Not shown electrodes in the recess 12 (see FIG. 4c) of the drop sensor come into electrical contact with electrodes 20, 21, when the protuberance 11 is inserted in recess 12. The specific resistance of the resistance element is sensed and is transmitted to the flow control device 5 (see FIGS. 1a-1c) for characterizing the drip chamber as regards the drop size generated by the specific drip chamber.

Alternatively, an electrical circuit containing information as regards drop size and production series number, etc. may be embedded in protuberance 11 between the electrodes 20 and 21 instead of the resistance element 22.

Referring now to FIGS. 8a-8d, two spaced electrodes 23, 24 forming a capacitor are provided on the sensor 4 in an annular recess 4a having protruding annular edges 4b and 4c, and an electrically conductive ring 25 is arranged on an annular ring shaped thickened portion of the drip chamber 1 having annular edges 25a and 25b.

When the drip chamber ring 25 is inserted in the recess 4a so that the edges 4c and 4b snap around the edges 25a and 25b and axially fixate the drip chamber relative to the drop sensor, the ring 25 will be located between the capacitor electrodes 23, 24 and determine the capacity thereof as a function of the width of the ring 25 as indicated in FIGS. 8c and 8d where the ring 25 is shown with different widths. Hereby information based on the width of the ring 25 regarding the character of the drip chamber can be transmitted to the flow regulating device 5 (see FIG. 3).

Referring now to FIG. 9a, a bar code 26 is located on the surface of the drip chamber 1, and a bar code reader 27 is provided on the drop sensor 4.

Prior to attaching the drop sensor 4 to the drip chamber 1, the bar code 26 is read by passing the bar code reader 27 along the bar code 26, and the information on the bar code 26 is transmitted to the regulating device 5.

Referring now to FIG. 9b, an RFID tag 26 is located on the surface of the drip chamber 1, and an RFID sensing coil 29 is provided on the drop sensor 4.

Prior to or after attaching the drop sensor 4 to the drip chamber 1, the RFID tag is sensed by the coil 29, and the information on the tag 28 is transmitted to the regulating device 5.

Referring now to FIG. 9c, the embodiment of FIG. 9b has been supplemented with a bar code 30 arranged on top of the RFID tag 28 and a bar code reader 27 has also been provided. The drop sensor of FIG. 9c can also be employed with a drip chamber having only one of a bar code and an RFID tag.

Referring now to FIG. 9d, a QR code 31 is located on the surface of the drip chamber 1, and an image generating means in the form of a digital camera 32 is provided on the drop sensor 4. Prior to attaching the drop sensor 4 to the drip chamber 1, the QR code is photographed by the camera 32 and the digital image is transmitted to the regulating device 5 where the information embedded in the image is processed.

Apart from ensuring that the drop sensor and the drip chamber are compatible, important advantages may be achieved by the above features, particularly if the flow regulating system comprises wire-less communication means such as Wi-Fi, Sim card, Bluetooth, radio, infrared etc. and computing means.

The drip chamber may be recognized by the system by geometry, colour code, markings, resistance, electrical capacity, etc. This can be used to ensure compatibility with the rest of the system, or register drop size for flow rate control and so on.

By means of the information on the RFID tag, bar code and QR code as well as digital images of the medicine label on the liquid supply container gathered by the digital camera, the function of the flow regulating device may be controlled, and by wire-less communication with a database, a PC or a responsible doctor, the correct treatment may be programmed and/or registered for documentation purposes.

According to the invention, one or more of the RFID sensing means, the bar code reader and the digital camera may be located on or in the flow regulating device 5 as indicated by the a camera lens 33 in FIG. 10.

1. A combination of
a drip chamber for use in a drip infusion set of the type comprising a liquid supply connected to said drip chamber upstream thereof and an injection needle connected through a tube to said drip chamber downstream thereof,
and a drop sensor adapted for being arranged adjacent said drip chamber for sensing the passage of drops through said drip chamber,
wherein said drip chamber is provided with characterization means for characterizing said drip chamber, and said drop sensor is provided with corresponding identification means for identifying and recognizing said characterization means.

2. A combination according to claim 1, wherein said characterization means and said corresponding identification means are chosen from a group of characterization means and corresponding identification means comprising:
a) a specific drip chamber surface, and
a corresponding drop chamber surface adapted for engaging said drip chamber surface, said drip chamber surface deviating from a circular cylindrical configuration,
b) a specifically shaped protuberance of one of said drip chamber and said drop sensor and
a correspondingly shaped recess of the second of said drip chamber and said drop sensor adapted for receiving said protuberance,
c) an electrically conductive element forming part of said drip chamber and having a specific resistance between first two electrical contacts thereof and
corresponding second electrical contacts forming part of said drop sensor, said drop sensor being provided with means to measure said resistance when said first and second contacts are interconnected,
d) an electrical circuit containing specific information regarding said drip chamber and forming part of said drip chamber, and
sensing means for sensing said specific information and forming part of said drop sensor,
e) an RFID tag containing specific information regarding said drip chamber and forming part of said drip chamber, and
RFID sensing means for sensing said RFID tag and forming part of said drop sensor,
f) a bar code containing specific information regarding said drip chamber and forming part of said drip chamber, and
a bar code reader for reading said bar code and forming part of said drop sensor,
g) a QR code containing specific information regarding said drip chamber and forming part of said drip chamber, and
image generating means such as a camera for generating a digital image of said QR code and forming part of said drop sensor,
h) two capacitor electrodes constituting a capacitor element and forming part of said drop sensor, and
a corresponding electrically conductive element forming part of said drip chamber and adapted for being located between said capacitor electrodes when said drop sensor engages said drip chamber such that the electrical capacity of said capacitor element depends on the configuration of said electrically conductive element when said electrically conductive element is located between said two capacitor electrodes. said drop sensor being provided with means for measuring said electrical capacity, and
i) visually identifiable means such as shape, colour, markings and the like forming part of said drip chamber, and
image generating means such as a camera for generating a digital image of said visually identifiable means and forming part of said drop sensor.

3. A drip chamber for use in a drip infusion set of the type comprising a liquid supply connected to said drip chamber upstream thereof and an injection needle connected to said drip chamber downstream thereof, said drip chamber being provided with the characterization means according to claim 1, wherein the outer surface of said drip chamber surface deviates from a circular cylindrical configuration.

4. A drop sensor adapted for being arranged adjacent a drip chamber for use in a drip infusion set of the type comprising a liquid supply connected to said drip chamber upstream thereof and an injection needle connected to said drip chamber downstream thereof, said drop sensor being provided with the identification means according to claim 1.

5. A combination according to claim 1 and further comprising a liquid flow control device comprising a valve adapted for controlling the flow of said liquid through said tube.

6. A liquid flow control system for use with drip infusion sets of the type comprising a liquid supply, a drip chamber downstream of the liquid supply for forming liquid drops and a tube connecting said drip chamber with an injection needle, said system comprising:
a drop sensor adapted for being arranged adjacent said drip chamber for sensing the passage of drops through said drip chamber,
a valve adapted for controlling the flow of said liquid through said tube,
valve activating means for activating said valve,
electrical circuits for registering the rate of drop formation and controlling said valve activating means,
a battery for powering said drop sensor, said electrical circuits and said valve activating means, and
identification means for identifying features relevant to the correct functioning of said system.

7. A system according to claim 6, wherein said identification means are chosen from a group of identification means comprising:
F) image generating means such as a digital camera for generating a digital image,
G) a bar code reader for reading a bar code, and
H) RFID sensing means for sensing an RFID tag.
I) Means to measure a resistance, and
J) Means to measure an electrical capacity.

8. A system according to claim 6, wherein said features are chosen from a group of features comprising:
10) a colour code on the drip chamber,
11) markings on the drip chamber,
12) a specific shape of the drip chamber,
13) a resistance in an electrical conductor forming part of said drip chamber,
14) an electrical capacity in a capacitor forming part of said drip chamber,
15) an RFID tag,
16) a bar code,
17) a QR code, and
18) printed information.

9. A system according to claim 6, wherein the system further comprises wire-less communication means.

10. A method of ensuring correct function of a drip infusion set of the type comprising a liquid supply connected to a drip chamber upstream thereof and an injection needle connected to said drip chamber downstream thereof as well as a drop counter for sensing the passage of drops through said drip chamber for counting the drops in said drip chamber, said method comprising the following steps:
a) providing:
a valve adapted for controlling the flow of said liquid through said tube,
valve activating means for activating said valve,
electrical circuits for registering the rate of drop formation and controlling said valve activating means,
a battery for powering said drop sensor, said electrical circuits and said valve activating means,
b) providing said drip chamber with characterization means according to claim 2,
c) providing said drop sensor with identification means according to claim 2,
d) transmitting data generated by said identification means to said electrical circuits, and
e) causing said electrical circuits to control said valve activating means based on said data.

11. A method according to claim 10 comprising the further steps of:
a) providing wire-less communication means, and
b) causing said wire-less communication means to transmit and receive data including said data generated by said identification means.
